(19) United States
(12) Patent Application Publication
(10) Pub. No.: US 2020/0187908 A1
(43) Pub. Date: Jun. 18, 2020

(54) METHOD AND SYSTEMS FOR TOUCHSCREEN USER INTERFACE CONTROLS

(71) Applicant: General Electric Company, Schenectady, NY (US)

(72) Inventors: Heinz Schmied, Mondsee (AT); Andreas Doninger, Frankenburg am Hausruck (AT)

(21) Appl. No.: 16/224,491

(22) Filed: Dec. 18, 2018

(51) Int. Cl. A61B 8/00; G06F 3/0488

(52) U.S. Cl. CPC A61B 8/469; A61B 8/465; G06F 3/04886

(57) ABSTRACT

Various methods and systems are provided for imaging system user interfaces. In one embodiment, a method comprises displaying, via a touchscreen, a first virtual button, displaying a menu comprising a plurality of virtual buttons corresponding to actions responsive to detecting a finger pressing the first virtual button via the touchscreen, performing an action responsive to the finger being released from the touchscreen at a second virtual button associated with the action, and updating the display of the first virtual button to indicate the action. In this way, an operator of an imaging system may easily access a potentially large plurality of imaging modes and actions relating to imaging via a touchscreen during a scan, thereby extending the operator's ability to control the ultrasound imaging system in a reduced amount of time.

### FIELD

Embodiments of the subject matter disclosed herein relate to ultrasound imaging.

### BACKGROUND

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device. In one example, the display device may be a touch-sensitive display, also referred to as a touchscreen. A user may interact with the touchscreen to analyze the displayed image. For example, a user may use their fingers on the touchscreen to position a region of interest (ROI), place measurement calipers, or the like.

### BRIEF DESCRIPTION

In one embodiment, a method comprises displaying, via a touch-sensitive display device, a first virtual button, displaying a menu comprising a plurality of virtual buttons corresponding to actions associated with the first virtual button responsive to detecting a finger pressing the first virtual button via the touch-sensitive display device, performing an action of the actions responsive to the finger being released from the touch-sensitive display device at a second virtual button of the plurality of virtual buttons associated with the action, and updating the display of the first virtual button to indicate the action. In this way, an operator of an ultrasound imaging system may easily access a potentially large plurality of imaging modes and actions relating to ultrasound imaging via a touchscreen during a scan, thereby extending the operator's ability to control the ultrasound imaging system in a reduced amount of time.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system according to an embodiment;

FIG. 2 shows a high-level flow chart illustrating an example method for changing an imaging mode during an imaging session according to an embodiment;

FIG. 3 shows a high-level flow chart illustrating an example method for displaying imaging mode options for user selection according to an embodiment;

FIG. 4 shows an example touch-sensitive display device with virtual buttons according to an embodiment;

FIG. 5 shows an example touch-sensitive display device with a displayed menu according to an embodiment;

FIG. 6 shows an example touch-sensitive display device with an activated virtual button according to an embodiment;

FIG. 7 shows an example touch-sensitive display device with a selection of an alternate imaging mode from a displayed menu according to an embodiment;

FIG. 8 shows an example touch-sensitive display device with an activated virtual button for a selected imaging mode according to an embodiment;

FIG. 9 shows an example touch-sensitive display device with a displayed menu including sorted imaging modes according to an embodiment; and

FIG. 10 shows an example touch-sensitive display device with a deactivated virtual button according to an embodiment.

### DETAILED DESCRIPTION

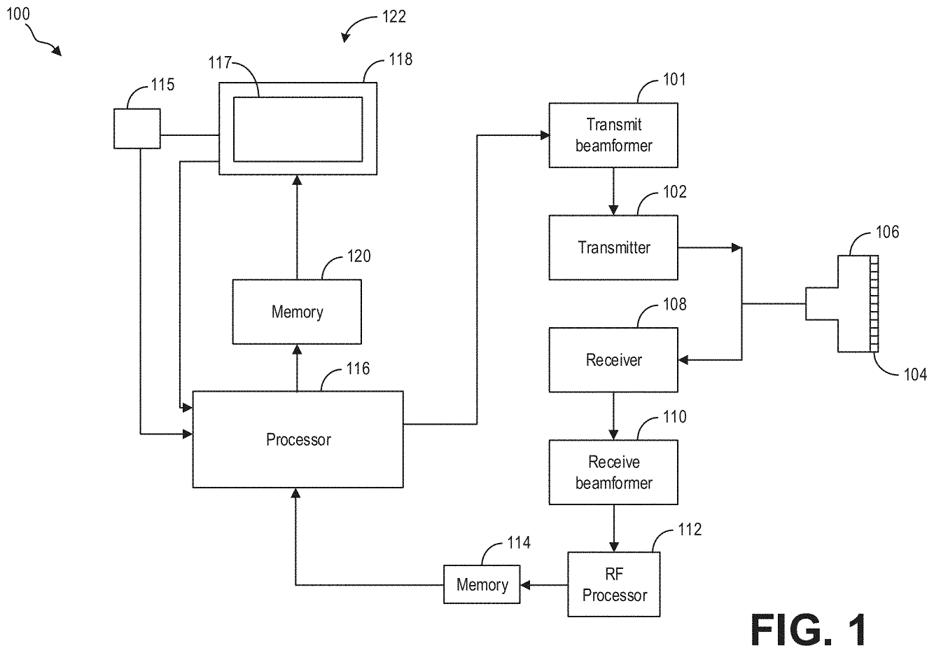

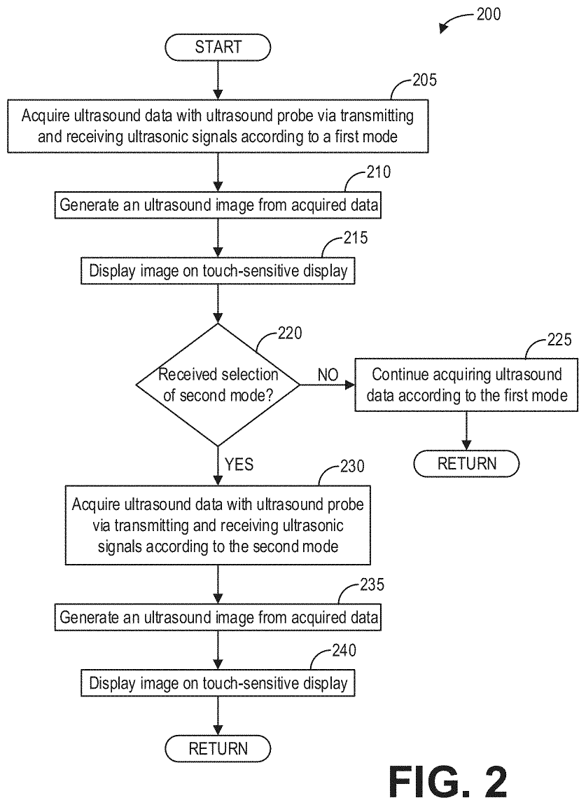

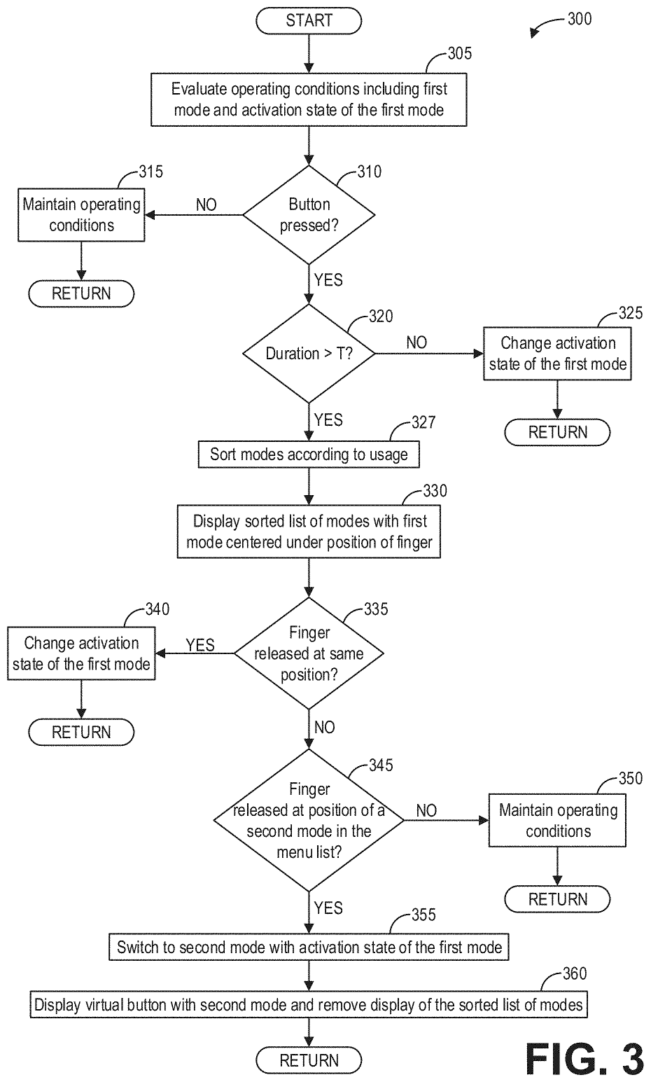

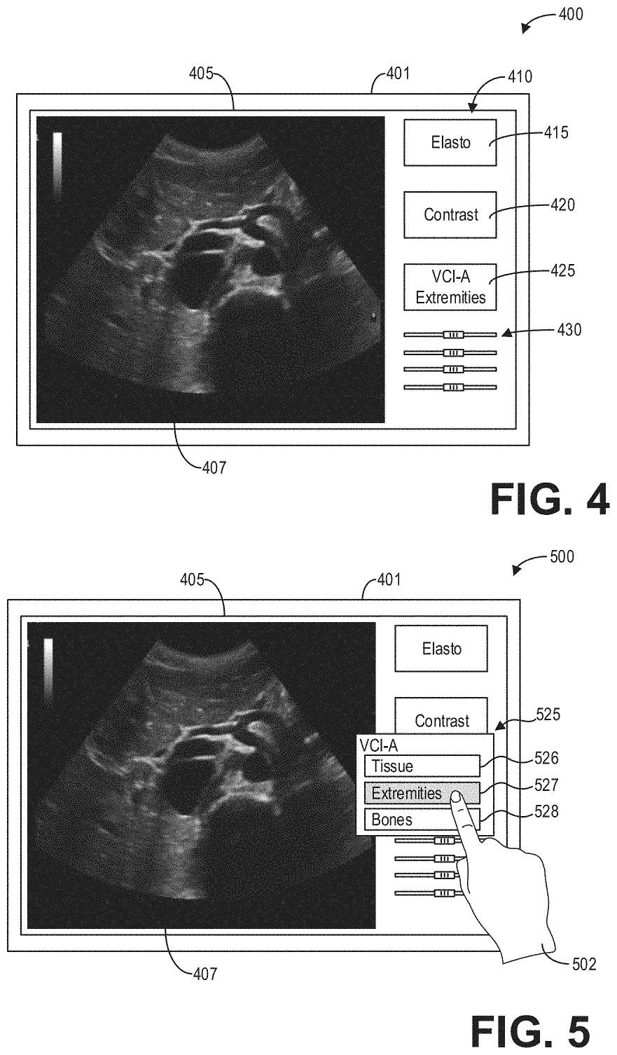

The following description relates to various embodiments of ultrasound imaging, such as the ultrasound imaging system shown in FIG. 1. In particular, systems and methods for touchscreen user interface controls are provided. A method for ultrasound imaging, such as the method depicted in FIG. 2, includes switching from a first imaging mode to a second image mode responsive to an operator of the ultrasound imaging system selecting the second image mode via a touchscreen. The display area of the touch screen may be limited or constrained due to size limitations, and thus the plurality of imaging options, actions, and modes available to the operator for an ultrasound scan may not be easily accessible, especially during a scan when the operator may be occupied with handling an ultrasound probe. A method for providing quick access to the plurality of imaging modes available to an operator, such as the method depicted in FIG. 3, therefore includes displaying a menu comprising a sorted list of imaging modes or other relevant actions responsive to the operator pressing a virtual button on the touchscreen. As depicted by the example display outputs in FIGS. 4-10, the imaging modes and actions may be sorted according to usage, such that recently used or more regularly used imaging modes and actions are quickly accessible. Further, the virtual button(s) allow the operator to quickly activate or deactivate the imaging modes. In this way, the number of interaction steps and therefore the interaction time for activating a touch button or accessing different controls is minimized.

FIG. 1 illustrates a block diagram of a system 100 according to one embodiment. In the illustrated embodiment, the system 100 is an imaging system, and more specifically, an ultrasound imaging system. However, it is understood that embodiments set forth herein may be implemented using other types of medical imaging modalities (e.g., MR, CT, PET/CT, SPECT, and so on). Furthermore, it is understood that other embodiments do not actively acquire medical images. Instead, embodiments may retrieve image data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart, or may comprise a handheld device.

In the illustrated embodiment, the system 100 includes a transmit beamformer 101 and a transmitter 102 that drive elements 104, such as piezoelectric crystals, within a transducer array, or probe, 106 to emit pulsed ultrasonic signals into a body or volume (not shown) of a subject. The elements 104 and the probe 106 may have a variety of geometries. For example, the probe 106 may be a one-dimensional transducer array probe or a two-dimensional matrix transducer array probe. The ultrasonic signals are back-scattered from structures in the body, such as blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that performs beamforming and outputs an RF signal or ultrasound data. The RF signal or ultrasound data is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage).

According to some embodiments, the probe 106 may contain electronic circuitry to do all or part of the transmit and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system.

The system 100 also includes a controller or processor 116 configured to control operation of the system 100, including the transmit beamformer 101, the transmitter 102, the receiver 108, and the receive beamformer 110. The processor 116 is in electronic communication with the probe 106. For purposes of this disclosure, the term “electronic communication” may be defined to include both wired and wireless communications. The processor 116 may control the probe 106 to acquire data. The processor 116 controls which of the elements 104 are active and the shape of a beam emitted from the probe 106.

The processor 116 may include a central processor (CPU) according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board.

The processor 116 is adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. To that end, the processor 116 may include an image processing module (not shown) that receives image data (e.g., ultrasound signals in the form of RF signal data or IQ data pairs) and processes image data. For example, the image processing module may process the ultrasound signals to generate slices or frames of ultrasound information (e.g., ultrasound images) for displaying to the operator. When the system 100 is an ultrasound system, the image processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler, C-scan, and elastography. The generated ultrasound images may be two-dimensional (2D) or three-dimensional (3D). When multiple two-dimensional images are obtained, the image processing module may also be configured to stabilize or register the images. The image lines and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may be provided that reads the image volumes from a memory and displays an image in real time while a procedure is being carried out on a patient. A video processor module may store the images in an image memory, from which the images are read and displayed.

As mentioned above, acquired ultrasound information may be processed in real-time during an imaging session (or scanning session) as the echo signals are received. Additionally or alternatively, the ultrasound information may be stored temporarily in a buffer or memory 114 during an imaging session and processed in less than real-time in a live or off-line operation. An image memory 120 is included for storing processed slices of acquired ultrasound information that are not scheduled to be displayed immediately. The image memory 120 may comprise any known data storage medium, for example, a permanent storage medium, removable storage medium, and the like. Additionally, the image memory 120 may comprise a non-transitory storage medium.

In operation, an ultrasound system may acquire data, for example, volumetric data sets by various techniques (e.g., 3D scanning, real-time 3D imaging, volume scanning, 2D scanning with probes having positioning sensors, freehand scanning using a voxel correlation technique, scanning using 2D or matrix array probes, and the like). Ultrasound images of the system 100 may be generated from the acquired data at the processor 116 and displayed to the operator or user on a display device 118.

The processor 116 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In some embodiments, the user interface 122 may also include one or more user interface input devices 115, such as a physical keyboard, mouse, and/or touchpad. In some embodiments, the user interface input device 115 comprises a touchpad communicatively coupled to the processor 116 and the display device 118, such that when a user moves a finger, glove, or stylus across the face of the touchpad, a cursor atop the display area 117 moves in a corresponding manner. In other embodiments, the display device 118 comprises a touch-sensitive display (e.g., a touchscreen) that can detect a presence of a touch from the operator on the display area 117 and can also identify a location of the touch in the display area 117. The touch may be applied, for example, by at least one of an individual's hand or finger, a glove, a stylus, and the like. As such, the touch-sensitive display may also be characterized as an input device that is configured to receive inputs from the operator. The display device 118 also communicates information from the processor 116 to the operator by displaying the information to the operator. The display device 118 is configured to present information to the operator during the imaging session. For example, the information presented may include ultrasound images, graphical elements, user-selectable elements, and other information (e.g., administrative information, personal information of the subject, and the like).

FIG. 2 shows a high-level flow chart illustrating an example method 200 for changing an imaging mode during an imaging session according to an embodiment. In particular, method 200 relates to adjusting the acquisition and/or image processing settings during a scan responsive to a selection of an imaging mode via a touchscreen user interface. Method 200 is described with regard to the systems and components of FIG. 1, though it should be appreciated that the method may be implemented with other systems and components without departing from the scope of the present disclosure. Method 200 may be implemented as executable instructions in non-transitory memory, such as memory 120, and executed by a processor, such as processor 116, of the system 100.

Method 200 begins at 205. At 205, method 200 acquires ultrasound data with an ultrasound probe via transmitting and receiving ultrasonic signals according to a first mode. The first mode comprises a first imaging mode including one or more of transmit settings, receive settings, and/or image processing settings. At 210, method 200 generates an ultrasound image from the acquired data. The ultrasound image may be generated according to the first mode, in some examples. Continuing at 215, method 200 displays the ultrasound image on a touch-sensitive display, such as the display device 118.

At 220, method 200 determines if a selection of a second mode is received. A selection of a second mode is received if method 200 detects the presence of a finger or stylus, for example, in an area of the display area 117 of the display device 118 corresponding to a virtual button associated with the second mode. As discussed further herein with regard to FIG. 3, the second mode may be associated with the first mode. The display device 118 may dynamically display a sorted menu of imaging modes responsive to the operator touching the virtual button on the display device 118. The operator may then select the second mode from the sorted menu of imaging modes.

If a selection of a second mode is not received (“NO”), method 200 proceeds to 225. At 225, method 200 continues acquiring ultrasound data according to the first mode. Method 200 then returns. Thus, if the operator does not touch the virtual button to select a second imaging mode, method 200 continues acquiring ultrasound data and generating images according to the first imaging mode.

However, referring again to 220, if a selection of a second mode is received (“YES”), method 200 continues to 230. At 230, method 200 acquires ultrasound data with the ultrasound probe via transmitting and receiving ultrasonic signals according to the second mode, wherein the second mode comprises a second imaging mode including one or more of transmit settings, receive settings, and/or image processing settings.

At 235, method 200 generates an ultrasound image from the ultrasound data acquired at 230. Method 200 may generate the ultrasound image according to the second mode, in some examples. Continuing at 240, method 200 displays the ultrasound image generated at 235 on the touch-sensitive display. Method 200 then returns. Thus, by using the method for switching imaging modes via a touch-sensitive display as described herein, an operator of an ultrasound imaging system such as the system 100 may easily select imaging modes during a scan for instant use.

To simplify the process of selecting an imaging mode from a potentially large plurality of imaging modes, especially during a scan when the hands of the operator may be occupied with handling the probe 106, for example, and given the limited amount of space in the display area 117 of the display device 118, a method for displaying imaging mode options to the operator may include dynamically displaying a sorted list of imaging modes or other imaging actions responsive to the operator touching a virtual button on the display device 118. As an example, FIG. 3 shows a high-level flow chart illustrating an example method 300 for displaying imaging mode options for user selection according to an embodiment. In particular, method 300 relates to displaying a sorted menu responsive to a virtual button being pressed. Method 300 is described with regard to the systems and components of FIG. 1, though it should be appreciated that the method may be implemented with other systems and components without departing from the scope of the present disclosure. For example, method 300 may be stored as executable instructions in non-transitory memory, such as memory 120, and executed by a processor, such as processor 116, of the system 100.

Method 300 begins at 305. At 305, method 300 evaluates operating conditions including a first mode associated with a virtual button and an activation state of the first mode. The first mode comprises, for example, an imaging mode associated with the virtual button, wherein the virtual button is displayed via a touch-sensitive display device such as the touch-sensitive display device 118. Further, the first mode may comprise a most-recently-used imaging mode associated with the virtual button. Additionally or alternatively, the first mode may comprise a default imaging mode associated with the virtual button. For example, upon initializing a scanning session with the system 100, the virtual button may indicate a default imaging mode associated with the virtual button. The default imaging mode may be predetermined, in some examples, though in other examples the default imaging mode may comprise an imaging mode associated with the virtual button that is used more often than other imaging modes associated with the virtual button. Furthermore, it should be appreciated that method 300 may be executed repeatedly or continuously during a scanning session. Therefore, while executing method 300 during a given scanning session, for example, the first mode may comprise an imaging mode most recently used during the scanning session. The activation state of the first mode may comprise an activated state or a deactivated state, as an illustrative example. Thus, method 300 determines the current operating conditions of the system 100, and in particular method 300 determines what the first mode or the currently-selected mode associated with the virtual button as well as whether the first mode is activated or deactivated.

Continuing at 310, method 300 determines if the virtual button is pressed. The virtual button is pressed if the display device 118 detects a finger, for example, touching an area of the display area 117 on the display device 118 associated with the virtual button.

If the virtual button is not pressed (“NO”), method 300 continues to 315. At 315, method 300 maintains the operating conditions. That is, method 300 maintains the first mode displayed via the virtual button on the display device 118 and further maintains the activation state of the first mode. Method 300 then returns. By repeatedly executing method 300, method 300 may evaluate whether the virtual button is pressed, and maintains operating conditions associated with the virtual button until the virtual button is pressed.

Thus, referring again to 310, if a virtual button is pressed (“YES”), method 300 continues to 320. At 320, method 300 determines if the duration of the virtual button being pressed is greater than a threshold T. The threshold T may be predetermined to establish whether the virtual button is being held. For example, if the duration is not greater than the threshold T (“NO”), method 300 continues to 325, whereupon method 300 changes the activation state of the first mode. For example, if the activation state of the first mode at 305 is activated, method 300 deactivates the first mode. Conversely, if the activation state of the first mode at 305 is deactivated, method 300 activates the first mode. Method 300 then returns after changing the activation state of the first mode. Thus, pressing the virtual button for a duration less than the threshold T changes the activation state of the first mode.

However, referring again to 320, if the duration is greater than the threshold T (“YES”), method 300 continues to 327. At 327, method 300 sorts the modes associated with the virtual button according to usage. In one example, method 300 sorts the modes according to recent usage. For example, for a list of imaging modes, the imaging modes may be sorted according to the imaging modes most recently used during a scan. As another example, method 300 may sort the modes according to the number of uses per mode. For example, for a list of imaging modes, the imaging modes used more frequently may be sorted to the top of the list, while imaging modes used more rarely may be sorted to the bottom of the list. As the first mode is the mode currently displayed via the virtual button, method 300 may exclude the first mode from the list of modes when sorting the modes.

After sorting the list of modes according to usage, method 300 continues to 330. At 330, method 300 displays the sorted list of modes with the first mode centered under the position of the finger pressing the virtual button. The display area indicating each mode displayed comprises a virtual button in the display area 117 of the display device 118. Therefore, the operator may drag the finger from the virtual button associated with the first mode to a virtual button associated with another mode in the sorted list of modes, and release the finger at a virtual button associated with a second mode to select the second mode. The display of the sorted list of modes comprises a pop-up menu in the display area 117. That is, the sorted list of modes is not displayed in the display area 117 until the finger is pressing the virtual button for a duration greater than the threshold T. In this way, the operator may quickly access the modes in the sorted list of modes by pressing the virtual button, without the need to navigate through multiple menus.

Furthermore, method 300 may display the list of modes in alternating order from the first mode. For example, as mentioned above, the first mode may be excluded from the sorted list of modes, as the first mode is currently displayed via the virtual button and is displayed under the position of the finger pressing the virtual button. The first mode in the sorted list of modes may thus be displayed under the first mode, while the second mode in the sorted list of modes may be displayed above the first mode. Therefore the most popular mode in the list of modes (excluding the first mode) may be displayed adjacent to and below the first mode, or alternatively the most recently used mode in the list of modes (excluding the first mode) may be displayed adjacent to and below the first mode, while the second most popular mode or the second most-recently-used mode may be displayed above the first mode. Similarly, the third most popular mode or the third most-recently-used mode may be displayed under the most popular mode or the most-recently-used mode, the fourth most popular mode or the fourth most-recently-used mode may be displayed above the second most popular mode or the second most-recently-used mode, and so on. As another example, method 300 may display the list of modes in descending sorted order below the first mode. In some examples, method 300 may display the sorted list of modes radially rather than linearly, where the distance of the modes from the first mode is based on the position of the modes in the sorted list. Therefore more popular or more recently-used modes may be positioned closer to the first mode, while less popular or less recently-used modes are positioned further from the first mode.

At 335, method 300 determines if the finger is released at the same position. If the finger is released at the same position (“YES”), then method 300 considers the first mode which is at the position to be selected by the operator. Method 300 continues to 340, whereupon method 300 changes the activation state of the first mode. For example, if the first mode is activated at 305, method 300 deactivates the first mode at 340. Similarly, if the first mode is deactivated at 305, method 300 activates the first mode at 340. Method 300 then returns. Thus, the operator may press a finger to the virtual button to view the sorted list of modes, and release the finger without moving it away from the first mode to change the activation state of the first mode, similar to pressing the virtual button without holding the finger at the virtual button as occurs at 325.

However, referring again to 335, if the finger is not released at the same position (“NO”), the finger is released at a position other than the first mode. Method 300 then continues to 345. At 345, method 300 determines if the finger is released at a position of a second mode in the displayed menu list. If the finger is not released at a position of a second mode in the displayed menu list (“NO”), then the finger is released at a position away from the modes of the displayed menu list. Method 300 continues to 350. At 350, method 300 maintains the operating conditions. That is, method 300 does not affect the operating conditions by switching modes or changing the activation state of a mode. Method 300 then returns. In this way, an operator may choose to not change the activation state of the first mode as well as not choosing a second mode from the displayed list of modes, despite pressing the virtual button at 310.

However, referring again to 345, if the finger is released at a position of a second mode in the displayed menu list (“YES”), method 300 continues to 355. The release of the finger at the position of the second mode comprises a selection of the second mode. Therefore, at 355, method 300 switches to the second mode with the activation state of the first mode. That is, the activation state of the first mode determined at 305 is applied to the second mode. For example, if the first mode was activate at 305, method 300 switches to the second mode in the active state. Similarly, if the first mode was not active at 305, method 300 switches to the second mode in a deactivated state. Continuing at 360, method 300 displays the virtual button with the second mode and removes the display of the sorted list of modes. Method 300 then returns. Thus, the operator may quickly select a second mode from a potentially large list of modes via a touch-sensitive display, without the list of modes being continuously displayed in the display area of the touch-sensitive display. Further, by sorting the modes according to usage, the imaging mode most likely desired by the operator may be easily accessible.

As an illustrative example, FIGS. 4-10 show example display outputs during a selection by an operator of a different imaging mode. In particular, FIG. 4 shows an example display output 400 on a touch-sensitive display device 401. The touch-sensitive display device 401 may comprise the display device 118, for example, while the display area 405 may correspond to the display area 117 of FIG. 1. The display area 405 may display an ultrasound image 407, as well as a plurality of user-selectable virtual inputs 410. The plurality of user-selectable virtual inputs 410 may include, as non-limiting and illustrative examples, a virtual button 415 for controlling an elastography (“Elasto”) imaging mode, a virtual button 420 for controlling a contrast imaging mode, a virtual button 425 for controlling a volume contrast imaging (VCI) in a particular plane (e.g., the A plane, or VCI-A), a plurality of sliders 430 for adjusting settings, and so on. It should be appreciated that additional information as well as additional or alternative user-selectable user inputs may be displayed in the display area 405. The plurality of user-selectable virtual inputs 410 may be selected or controlled responsive to the operator pressing and/or dragging a finger, stylus, or other suitable probe for interacting with the touch-sensitive display 118 to and/or across an area of the display area 405. For example, the operator may push and drag a finger at a slider of the plurality of sliders 430 to increase or decrease a parameter associated with the slider.

Further, as described hereinabove with regard to FIG. 3, pressing one or more of the virtual buttons 415, 420, and 425 may result in the display of a pop-up menu including a sorted list of actions or other imaging modes. For example, FIG. 5 shows a display output 500 when the operator 252 presses the virtual button 425 for controlling the VCI-A imaging modes. As depicted in FIG. 4, the virtual button 425 initially displays the VCI-A Extremities imaging mode. As depicted in FIG. 5, pressing the virtual button 425 prompts the pop-up menu 525 to display in the display area 405. The pop-up menu 525 includes a plurality of virtual buttons corresponding to various imaging modes associated with VCI-A, including a virtual button 526 for a Tissue imaging mode, a virtual button 527 for the Extremities imaging mode, and a virtual button 528 for a Bones imaging mode. The Tissue, Extremities, and Bones imaging modes specify a set of transmit, receive, and/or image processing parameters for optimally imaging tissue, extremities (e.g., hands, feet) and bones according to VCI-A, respectively, as illustrative examples.

As the Extremities imaging mode was initially displayed via the virtual button 425, the virtual button 527 for the Extremities imaging mode is displayed under the position where the operator 502 is pressing the display area 405. With regard to method 300 described herein above, the Extremities imaging mode thus corresponds to the first mode. Further, the list of modes includes the Bones imaging mode and the Tissue imaging mode. The Bones imaging mode comprises, for example, the most-recently-used or the most-often-used imaging mode of the list of modes, and therefore the virtual button 528 for the Bones imaging mode is displayed under the virtual button 527 for the Extremities imaging mode. The Tissue imaging mode comprises the second most-recently-used or the second most-often-used imaging mode of the list of modes, and therefore the virtual button 526 for the Tissue imaging mode is displayed above the virtual button 527 for the Extremities imaging mode.

It should be appreciated that the positions may be switched in some examples or according to the preference of the operator, such that the most-recently-used mode or the most-often-used mode may be positioned above the current mode while the second most-recently-used mode or the second most-often-used mode may be positioned below the current mode.

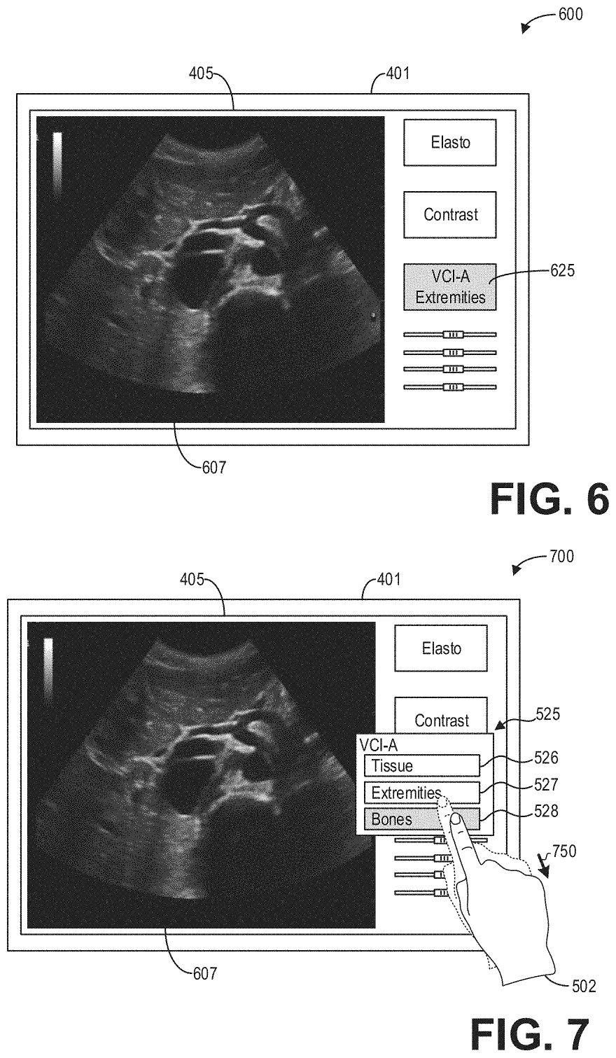

FIG. 6 shows the display output 600 when the operator 502 releases the finger from a position of the display area corresponding to the virtual button 527 for the Extremities imaging mode. As depicted, the display of the virtual button 625 is changed relative to the display of the virtual button 425 to reflect that the activation state of the corresponding imaging mode is changed. In particular, the virtual button 625 is shaded with respect to virtual button 425, thereby indicating that the VCI-A Extremities imaging mode is activated, though it should be appreciated that other methods for distinguishing the virtual button 625 from the virtual button 425, and therefore indicating the relative change in activation state, may be utilized. As the VCI-A Extremities imaging mode is activated, the ultrasound image 607 is acquired and/or generated according to the VCI-A Extremities imaging mode, and is therefore correspondingly different from the ultrasound image 407 depicted in FIGS. 4 and 5.

FIG. 7 shows a display output 700 depicting an example wherein the operator 502 presses the virtual button 625 to prompt the pop-up menu 525 to be displayed in the display area 405. As depicted, the operator 502 has dragged 750 the finger from the virtual button 527 for the Extremities imaging mode to the virtual button 528 for the Bones imaging mode.

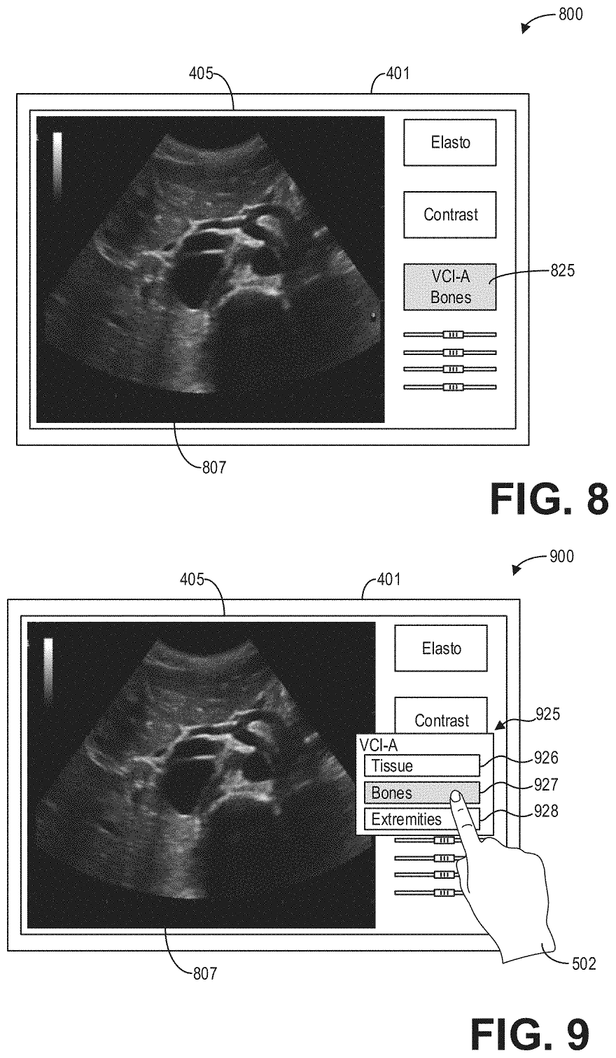

FIG. 8 shows the display output 800 depicting the display area 405 after the operator 502 releases the finger at the virtual button 528 for the Bones imaging mode. The virtual button 825 now depicts the VCI-A Bones imaging mode, and is shaded to indicate that the VCI-A Bones imaging mode is activated. As discussed herein above with regard to FIG. 3, the VCI-A Bones imaging mode is activated because the previous imaging mode associated with the virtual button 625 was activated prior to the operator 502 selecting the virtual button 528 for the Bones imaging mode.

FIG. 9 shows the display output 900 illustrating the display area 405 when the operator 502 presses the virtual button 825 depicted in FIG. 8. The display area 405 includes a pop-up menu 925 for the VCI-A imaging modes. As the Bones imaging mode is the current mode associated with the virtual button 825, the virtual button 927 for the Bones imaging mode is depicted in the center of the list of imaging modes, under the position of the finger of the operator 502 in the display area 405. Of the list of modes excluding the current mode (i.e., the Bones imaging mode), the modes are sorted according to usage as described herein. Thus, the virtual button 928 for the Extremities imaging mode is displayed under the virtual button 927 for the Bones imaging mode, as the Extremities imaging mode is the most-recently-used or the most-often-used mode of the list of modes including the Tissue imaging mode and the Extremities imaging mode, while the virtual button 926 for the Tissue imaging mode is displayed above the virtual button 927 for the Bones imaging mode.

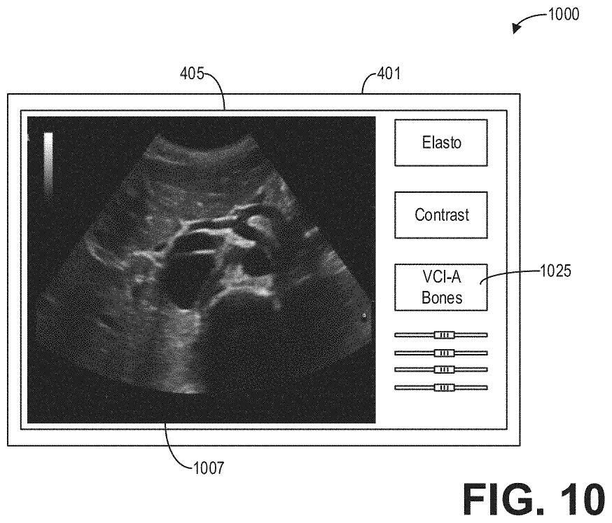

FIG. 10 shows the display output 1000 of the touch-sensitive display 401 after the operator 502 releases the finger from the position of the virtual button 927 for the Bones imaging mode depicted in FIG. 9. By releasing the finger from the virtual button 927 for the Bones imaging mode, the activation state of the imaging mode is thus changed. As depicted, the virtual button 1025 is un-shaded and depicts the Bones imaging mode, thereby indicating that the VCI-A Bones imaging mode is not active. Further, the ultrasound image 1007 is no longer acquired and/or generated according to the VCI-A Bones imaging mode, and therefore is different from the ultrasound image 807 depicted in FIGS. 8 and 9.

A technical effect of the disclosure includes the display of a menu including a plurality of actions responsive to detecting a virtual button being pressed. Another technical effect of the disclosure includes the switching of imaging modes responsive to an imaging mode being selected from a pop-up menu on a touch-sensitive display device during a scan. Yet another technical effect is the reduction of interactions with a touch-sensitive display device for controlling an imaging system. Another technical effect is the increase in speed for user interaction with a touchscreen. Yet another technical effect is the efficient spatial usage for a touch-sensitive display device-based user interface.

In one embodiment, a method comprises displaying, via a touch-sensitive display device, a first virtual button, displaying a menu comprising a plurality of virtual buttons corresponding to actions associated with the first virtual button responsive to detecting a finger pressing the first virtual button via the touch-sensitive display device, performing an action of the actions responsive to the finger being released from the touch-sensitive display device at a second virtual button of the plurality of virtual buttons associated with the action, and updating the display of the first virtual button to indicate the action.

In a first example of the method, the method further comprises removing the display of the menu responsive to the finger being released from the touch-sensitive display device. In a second example of the method optionally including the first example, the method further comprises sorting the actions according to usage of the actions, and displaying the plurality of virtual buttons in the menu according to the sorting of the actions. In a third example of the method optionally including one or more of the first and second examples, sorting the actions according to usage of the actions comprises sorting the actions according to recent usage of the actions. In a fourth example of the method optionally including one or more of the first through third examples, sorting the actions according to the usage of the actions comprises sorting the actions according to how often the actions are used. In a fifth example of the method optionally including one or more of the first through fourth examples, the first virtual button is associated with a first action prior to the finger pressing the first virtual button, the menu includes a virtual button for the first action positioned at a position of the finger pressing the touch-sensitive device, and displaying the plurality of virtual buttons according to the sorting of the actions comprises displaying a virtual button for a first sorted action below the virtual button for the first action, and displaying a virtual button for a second sorted action above the virtual button for the first action. In a sixth example of the method optionally including one or more of the first through fifth examples, the touch-sensitive display device is communicatively coupled to an ultrasound probe, and the actions comprise activation or deactivation of one or more ultrasound imaging modes associated with the first virtual button. In a seventh example of the method optionally including one or more of the first through sixth examples, the first virtual button indicates an activation state of a first ultrasound imaging mode, the action comprises a selection of a second ultrasound imaging mode, and performing the action comprises switching from the first ultrasound imaging mode to the second ultrasound imaging mode with the activation state of the first ultrasound imaging mode. In an eighth example of the method optionally including one or more of the first through seventh examples, the method further comprises, responsive to the finger releasing from the touch-sensitive device after pressing the first virtual button for less than a threshold duration, not displaying the menu and changing an activation state of the first virtual button.

In another embodiment, a method comprises detecting a finger pressing a touch-sensitive display device at a position of a first virtual button indicating a first imaging mode on the touch-sensitive display device, displaying a menu including a plurality of virtual buttons for a plurality of imaging modes, the plurality of virtual buttons including at least a virtual button for the first imaging mode and a virtual button for a second imaging mode, detecting the finger releasing from touch-sensitive display device at a position of the virtual button for the second imaging mode, discontinuing display of the menu, and updating the first virtual button to indicate the second imaging mode.

In a first example of the method, the method further comprises, prior to detecting the finger pressing the touch-sensitive display device: acquiring a first set of ultrasound data according to the first imaging mode; generating a first ultrasound image from the first set of ultrasound data; and displaying the first ultrasound image via the touch-sensitive display device. In a second example of the method optionally including the first example, the method further comprises, after detecting the finger releasing from the touch-sensitive display device at the virtual button for the second imaging mode: acquiring a second set of ultrasound data according to the second imaging mode; generating a second ultrasound image from the second set of ultrasound data; and displaying the second ultrasound image via the touch-sensitive display device. In a third example of the method optionally including one or more of the first and second examples, the method further comprises sorting the plurality of imaging modes according to usage, and displaying the plurality of virtual buttons for the plurality of imaging modes according to the sorting of the plurality of imaging modes. In a fourth example of the method optionally including one or more of the first through third examples, sorting the plurality of imaging modes according to usage comprises sorting the plurality of imaging modes according to how recently the plurality of imaging modes were used. In a fifth example of the method optionally including one or more of the first through fourth examples, sorting the plurality of imaging modes according to usage comprises sorting the plurality of imaging modes according to how often the plurality of imaging modes are used.

In yet another embodiment, a system comprises an ultrasound probe, a touch-sensitive display device, and a processor configured with instructions in non-transitory memory that when executed cause the processor to: acquire a first set of ultrasound data via the ultrasound probe according to a first imaging mode; display, via the touch-sensitive display device, a first virtual button corresponding to the first imaging mode; responsive to detecting a finger pressing the first virtual button via the touch-sensitive display device, display a menu comprising a plurality of virtual buttons for a plurality of imaging modes, the plurality of virtual buttons including at least a virtual button for the first imaging mode and a virtual button for a second imaging mode; deactivate the first imaging mode responsive to detecting the finger being released from the touch-sensitive display device at the virtual button for the first imaging mode; and acquire a second set of ultrasound data according to the second imaging mode responsive to detecting the finger being released from the touch-sensitive display device at the virtual button for the second imaging mode.

In a first example of the system, the processor is further configured with instructions in the non-transitory memory that when executed cause the processor to generate a first ultrasound image from the first set of ultrasound data according to the first imaging mode and display the first ultrasound image via the touch-sensitive display device prior to detecting the finger pressing the first virtual button. In a second example of the system optionally including the first example, the processor is further configured with instructions in the non-transitory memory that when executed cause the processor to generate a second ultrasound image from the second set of ultrasound data according to the second imaging mode, and display the second ultrasound image via the touch-sensitive display device. In a third example of the system optionally including one or more of the first and second examples, the processor is further configured with instructions in the non-transitory memory that when executed cause the processor to sort the plurality of imaging modes according to usage of the plurality of imaging modes, and displaying the plurality of virtual buttons in the menu according to the sorting of the plurality of imaging modes. In a fourth example of the system optionally including one or more of the first through third examples, the processor is further configured with instructions in the non-transitory memory that when executed cause the processor to remove the display of the menu from the touch-sensitive display device responsive to detecting the finger being released from the touch-sensitive display device.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
displaying, via a touch-sensitive display device, a first virtual button;
responsive to detecting a finger pressing the first virtual button via the touch-sensitive display device, displaying a menu comprising a plurality of virtual buttons corresponding to actions associated with the first virtual button;
performing an action of the actions responsive to the finger being released from the touch-sensitive display device at a second virtual button of the plurality of virtual buttons associated with the action; and
updating the display of the first virtual button to indicate the action.

2. The method of claim 1, further comprising removing the display of the menu responsive to the finger being released from the touch-sensitive display device.

3. The method of claim 1, further comprising sorting the actions according to usage of the actions, and displaying the plurality of virtual buttons in the menu according to the sorting of the actions.

4. The method of claim 3, wherein sorting the actions according to usage of the actions comprises sorting the actions according to recent usage of the actions.

5. The method of claim 3, wherein sorting the actions according to the usage of the actions comprises sorting the actions according to how often the actions are used.

6. The method of claim 3, wherein the first virtual button is associated with a first action prior to the finger pressing the first virtual button, wherein the menu includes a virtual button for the first action positioned at a position of the finger pressing the touch-sensitive device, and displaying the plurality of virtual buttons according to the sorting of the actions comprises displaying a virtual button for a first sorted action below the virtual button for the first action, and displaying a virtual button for a second sorted action above the virtual button for the first action.

7. The method of claim 1, wherein the touch-sensitive display device is communicatively coupled to an ultrasound probe, and wherein the actions comprise activation or deactivation of one or more ultrasound imaging modes associated with the first virtual button.

8. The method of claim 7, wherein the first virtual button indicates an activation state of a first ultrasound imaging mode, wherein the action comprises a selection of a second ultrasound imaging mode, and wherein performing the action comprises switching from the first ultrasound imaging mode to the second ultrasound imaging mode with the activation state of the first ultrasound imaging mode.

9. The method of claim 1, further comprising, responsive to the finger releasing from the touch-sensitive device after pressing the first virtual button for less than a threshold duration, not displaying the menu and changing an activation state of the first virtual button.

10. A method, comprising:
detecting a finger pressing a touch-sensitive display device at a position of a first virtual button indicating a first imaging mode on the touch-sensitive display device;
displaying a menu including a plurality of virtual buttons for a plurality of imaging modes, the plurality of virtual buttons including at least a virtual button for the first imaging mode and a virtual button for a second imaging mode;
detecting the finger releasing from touch-sensitive display device at a position of the virtual button for the second imaging mode;
discontinuing display of the menu; and
updating the first virtual button to indicate the second imaging mode.

11. The method of claim 10, further comprising, prior to detecting the finger pressing the touch-sensitive display device:
acquiring a first set of ultrasound data according to the first imaging mode;
generating a first ultrasound image from the first set of ultrasound data; and
displaying the first ultrasound image via the touch-sensitive display device.

12. The method of claim 11, further comprising, after detecting the finger releasing from the touch-sensitive display device at the virtual button for the second imaging mode:
acquiring a second set of ultrasound data according to the second imaging mode;
generating a second ultrasound image from the second set of ultrasound data; and
displaying the second ultrasound image via the touch-sensitive display device.

13. The method of claim 10, further comprising sorting the plurality of imaging modes according to usage, and displaying the plurality of virtual buttons for the plurality of imaging modes according to the sorting of the plurality of imaging modes.

14. The method of claim 13, wherein sorting the plurality of imaging modes according to usage comprises sorting the plurality of imaging modes according to how recently the plurality of imaging modes were used.

15. The method of claim 13, wherein sorting the plurality of imaging modes according to usage comprises sorting the plurality of imaging modes according to how often the plurality of imaging modes are used.

16. A system, comprising:
an ultrasound probe;
a touch-sensitive display device; and
a processor configured with instructions in non-transitory memory that when executed cause the processor to:
acquire a first set of ultrasound data via the ultrasound probe according to a first imaging mode;
display, via the touch-sensitive display device, a first virtual button corresponding to the first imaging mode;
responsive to detecting a finger pressing the first virtual button via the touch-sensitive display device, display a menu comprising a plurality of virtual buttons for a plurality of imaging modes, the plurality of virtual buttons including at least a virtual button for the first imaging mode and a virtual button for a second imaging mode;
deactivate the first imaging mode responsive to detecting the finger being released from the touch-sensitive display device at the virtual button for the first imaging mode; and
acquire a second set of ultrasound data according to the second imaging mode responsive to detecting the finger being released from the touch-sensitive display device at the virtual button for the second imaging mode.

17. The system of claim 16, wherein the processor is further configured with instructions in the non-transitory memory that when executed cause the processor to generate a first ultrasound image from the first set of ultrasound data according to the first imaging mode and display the first ultrasound image via the touch-sensitive display device prior to detecting the finger pressing the first virtual button.

18. The system of claim 17, wherein the processor is further configured with instructions in the non-transitory memory that when executed cause the processor to generate a second ultrasound image from the second set of ultrasound data according to the second imaging mode, and display the second ultrasound image via the touch-sensitive display device.

19. The system of claim 16, wherein the processor is further configured with instructions in the non-transitory memory that when executed cause the processor to sort the plurality of imaging modes according to usage of the plurality of imaging modes, and displaying the plurality of virtual buttons in the menu according to the sorting of the plurality of imaging modes.

20. The system of claim 16, wherein the processor is further configured with instructions in the non-transitory memory that when executed cause the processor to remove the display of the menu from the touch-sensitive display device responsive to detecting the finger being released from the touch-sensitive display device.
